(19) United States
(12) Patent Application Publication
(10) Pub. No.: US 2002/0001581 A1
(43) Pub. Date: Jan. 3, 2002

(54) Methods for transducing cells in blood vessels using recombinant AAV vectors

(76) Inventors: Carmel M. Lynch, Kirkland, WA (US); Randolph L. Geary, Winston-Salem, NC (US)

Correspondence Address: Catherine M. Polizzi, Morrison & Foerster LLP, 755 Page Mill Road, Palo Alto, CA, 94304, US

(21) Appl. No.: 09/938,200

(22) Filed: Aug. 23, 2001

Related U.S. Application Data
(63) Continuation of application No. 08/793,916, filed on Feb. 28, 1997, now abandoned.
Application No. 08/793,916 is a 371 of international application No. PCT/US97/03134, filed on Feb. 28, 1997.
(60) Provisional application No. 60/028,145, filed on Mar. 4, 1996.

(51) Int. Cl. A61K 48/00; C12N 15/861

(52) U.S. Cl. 424/93.21; 435/456

(57) ABSTRACT

Current techniques for expressing recombinant genes in cells of blood vessels following direct in vivo gene transfer are limited by attendant problems or limitations. Further, an effective method of transducing microvascular cells and/or cells involved in formation of new blood vessels (angiogenesis) has not been demonstrated. This invention provides methods of transducing cells in blood vessels using recombinant adeno-associated virus (AAV) vectors.

### TECHNICAL FIELD OF THE INVENTION

This invention relates to gene delivery, and more specifically to methods for transducing cells in blood vessels using recombinant adeno-associated virus (AAV) vectors.

### BACKGROUND OF THE INVENTION

Blood vessels, a major component of the cardiovascular system, form a network that permits blood to flow from the heart to cells throughout the body and back to the heart. In general, blood vessels are composed of three layers, the intima, media and adventitia. The intima in most undiseased arterial vessels generally comprises a luminal monolayer of endothelial cells. In larger arteries, the intima may contain smooth muscle cells; and, in diseases such as atherosclerosis, the intima may thicken with smooth muscle cells and inflammatory cells beneath the endothelial monolayer. The intima is separated from the media by the internal elastic lamina. The media generally comprises smooth muscle cells and their surrounding matrix material. In larger arteries, the media is composed of defined layers of smooth muscle cells separated by elastic fibers. The adventitia forms the outermost layer of the artery wall and is separated from the media by the external elastic lamina. The adventitia is generally composed of a loose matrix containing macrophages, fibroblasts, and other cell types, as well as the vasa vasorum (a rich network of adventitial microvessels).

Microvessels (which include arterioles, capillaries, venules, and the vasa vasorum) may differ from the general structural model outlined above in that the three layers may not be well defined. For instance, capillaries may comprise a monolayer of endothelial cells surrounded by a single layer of smooth muscle cells without any well-defined elastic layers.

Angiogenesis is the formation of new blood vessels. Angiogenesis occurs during fetal development of the vascular system, as well in a wide range of normal and postnatal pathological processes such as wound repair; neoplasia, and inflammation. [See, e.g., Diaz-Flores, 1994] Thus, a number of disease processes have abundant microvessels as key anatomical or pathological features. Such microvessels may contribute directly or indirectly to the development of specific illnesses or may represent a benign morphological feature. In a number of instances, microvessels and angiogenesis are believed to play central roles in the pathogenesis of disease. These include, for example, the growth and metastasis of many cancers, diabetic retinopathy, retinitis, heart failure, arthritis, psoriasis, ischemia, wound healing, hemangiomas and other vascular malformations. [See, e.g., Diaz-Flores, 1994] The cells comprising all tissues and organs require an extensive network of microvessels to support their normal function and viability. Since these microvessels may be functionally unique and distinct from larger blood vessels, microvessels provide unique targets for delivery of therapeutic polynucleotides to tissues. Specifically, by modifying gene expression in cells of microvessels within specific tissues, the tissue or organ may be enhanced in a positive way.

A number of studies have explored methods for delivering recombinant genetic material to vascular endothelial cells and smooth muscle cells and methods for modifying the process of angiogenesis, but these studies have revealed limitations which include the following: low efficiency of in vivo gene transfer into the target cells and limited expression of recombinant genes in vascular tissues (using plasmid DNA, liposomal vectors and retroviral vectors); transient vector expression in target tissues (using adenoviral vectors and liposomal vectors); significant immune response directed against certain viral vectors; and lack of specificity for cells, tissues and organs, where the generalized expression of a transgene or the reintroduction of cells genetically modified ex vivo could have unwanted or detrimental effects. [See, e.g., Clowes, 1994; Geary, 1993; Geary, 1994; Lemarchand, 1993; Lim, 1991; Lynch, 1992; Nabel, 1989; Nabel, 1990; Newman, 1995; Ojeifo, 1995; Plautz, 1991; Rome (1), 1994; Rome (2), 1994; Schwartz, 1990; and Zwiebel, 1989]

For example, although retroviral vectors are advantageous because of their general potential for stable long-term gene expression, target cell replication is required for stable provirus integration. However, most cells in the artery are generally quiescent. Quiescent cells can sometimes be isolated and induced to divide in order to achieve significant and efficient gene transfer; but, this method often requires that the cells be induced and genetically modified ex vivo and thereafter transplanted back into the donor host. Liposomal vector delivery systems have also been limited by inefficient uptake and transient episomal vector expression. Adenoviral vectors that have been efficient at infecting endothelial cells have been limited by transient episomal vector expression and by antigenicity limiting the efficacy of repeated applications.

Thus, to date, no studies have demonstrated efficient and significant expression of recombinant genes in cells of blood vessels following direct in vivo gene transfer without attendant problems or limitations such as those referred to above. Further, an effective method of transducing microvascular cells and/or cells involved in formation of new blood vessels has not been demonstrated.

### SUMMARY OF THE INVENTION

The present invention provides methods for transducing cells in blood vessels using recombinant AAV vectors. Preferred embodiments of the present invention include the following:

1. A method of transducing a cell in a blood vessel of an individual, comprising introducing a recombinant adeno-associated viral (rAAV) vector to a blood vessel of said individual in vivo.

2. A method of transducing a cell in a blood vessel according to embodiment 1, wherein said rAAV vector comprises a detectable marker gene.

3. A method of transducing a cell in a blood vessel according to embodiment 1, wherein said rAAV vector comprises a selectable marker gene.

4. A method of transducing a cell in a blood vessel according to embodiment 1, wherein said rAAV vector comprises a therapeutic gene.

5. A method of transducing a cell in a blood vessel according to embodiment 1, wherein said blood vessel is a microvessel selected from the group consisting of arteriole, capillary, venule, and adventitial microvessel.

6. A method of transducing a cell in a blood vessel according to embodiment 5, wherein said blood vessel is an adventitial microvessel.

7. A method of transducing a cell in a blood vessel according to embodiment 1, wherein said blood vessel is a microvessel and said cell is undergoing proliferation.

8. A method of transducing a cell in a blood vessel according to embodiment 1, wherein said cell is a primate cell.

9. A method of transducing a cell in a blood vessel according to embodiment 8, wherein said cell is a human cell.

10. A method of transducing a cell in a blood vessel according to embodiment 1, wherein said cell is a proliferating cell.

11. A method of transducing a cell in a blood vessel according to embodiment 10, wherein said cell is a proliferating microvascular cell.

12. A method of transducing a cell in a blood vessel according to embodiment 1, wherein said cell is a microvascular cell.

13. A method of transducing a cell in a blood vessel according to embodiment 12, wherein said cell is a microvascular endothelial cell.

14. A method of transducing a cell in a blood vessel according to embodiment 1, wherein said rAAV vector is introduced into the adventitia of an artery of said individual.

15. A transduced microvascular cell produced by introducing a recombinant adeno-associated viral (rAAV) vector to said microvascular cell.

16. A method for treating an individual for a disease condition, comprising transducing a cell in a blood vessel of said individual according to the method of embodiment 4.

### DETAILED DESCRIPTION OF THE INVENTION

Definitions

“Polynucleotide” refers to a polymeric form of nucleotides of any length, either ribonucleotides or deoxyribonucleotides, or analogs thereof. This term refers only to the primary structure of the molecule. Thus, double- and single-stranded DNA, as well as double- and single-stranded RNA are included. It also includes modified polynucleotides such as methylated or capped polynucleotides.

“Recombinant,” as applied to a polynucleotide, means that the polynucleotide is the product of various combinations of cloning, restriction and/or ligation steps, and other procedures that result in a construct that is distinct from a polynucleotide found in nature.

A “vector” refers to a recombinant plasmid or virus that comprises a polynucleotide to be delivered into a host cell, either in vitro or in vivo. The polynucleotide to be delivered may comprise a coding sequence of interest in gene therapy.

A “recombinant AAV vector” (or “rAAV vector”) refers to a vector comprising one or more polynucleotides of interest that are flanked by AAV inverted terminal repeat sequences (ITRs). One possible method of replicating and packaging a rAAV vector into infectious viral particles may be to introduce the rAAV vector into a host cell expressing the AAV “rep” and “cap” genes and infected with a suitable helper virus.

A “gene” refers to a polynucleotide or portion of a polynucleotide comprising a sequence that encodes a protein. For most situations, it is desirable for the gene to also comprise a promoter operably linked to the coding sequence in order to effectively promote transcription. Enhancers, repressors and other regulatory sequences may also be included in order to modulate activity of the gene, as is well known in the art. (See, e.g., the references cited below).

The terms “polypeptide,” “peptide,” and “protein” are used interchangeably to refer to polymers of amino acids of any length. These terms also include proteins that are post-translationally modified through reactions that include glycosylation, acetylation and phosphorylation.

AAV “rep” and “cap” genes, encoding replication and encapsidation proteins, respectively, have been found in all AAV serotypes examined, as described in various references cited herein. Typically, the rep and cap genes are found adjacent to each other in the AAV genome, and they are generally conserved among AAV serotypes.

A “helper virus” for AAV refers to a second virus that allows wild-type AAV, which is a defective parvovirus, to be replicated and packaged by a host cell. A number of such helper viruses have been identified in the art, including adenoviruses, herpesviruses, and poxviruses such as vaccinia.

“Packaging” as used herein refers to a series of subcellular events that results in the assembly and encapsidation of a rAAV vector. Thus, when a suitable vector plasmid is introduced into a packaging cell line under appropriate conditions, it can be replicated and assembled into a viral particle.

“Heterologous” means derived from a genotypically distinct entity from that of the rest of the entity to which it is compared. For example, a polynucleotide derived from one cell type and introduced by genetic engineering techniques into a different cell type is a heterologous polynucleotide which, when expressed, can encode a heterologous polypeptide. Similarly, a promoter that is removed from its native coding sequence and operably linked to a different coding sequence is a heterologous promoter.

“Promoter.” as used herein, refers to a genomic region that controls the transcription of a gene or coding sequence to which it is operably linked.

“Operably linked” refers to a juxtaposition, wherein the components so described are in a relationship permitting them to function in their intended manner. A promoter is operably linked to a coding sequence if the promoter controls transcription of the coding sequence. An operably linked promoter is usually in cis configuration with the coding sequence, but is not necessarily contiguous with it.

A “detectable marker gene” is a gene that allows cells carrying the gene to be specifically detected (e.g., distinguished from cells which do not carry the marker gene). A large variety of such marker genes are known in the art. Preferred examples thereof include detectable marker genes which encode proteins appearing on cellular surfaces, thereby facilitating simplified and rapid detection and/or cellular sorting. By way of illustration, the inventors utilized an alkaline phosphatase (“AP”) gene as a detectable marker, which allowed cells transduced with a vector carrying the AP gene to be detected based on expression of AP on the surface of transduced cells.

A “selectable marker gene” is a gene that allows cells carrying the gene to be specifically selected for or against, in the presence of a corresponding selective agent. By way of illustration, an antibiotic resistance gene can be used as a positive selectable marker gene that allows a host cell to be positively selected for in the presence of the corresponding antibiotic. A variety of positive and negative selectable markers are known in the art, some of which are described below.

A “therapeutic polynucleotide” or “therapeutic gene” refers to a nucleotide sequence that is capable, when transferred to an individual, of eliciting a prophylactic, curative or other beneficial effect in the individual.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins (such as IL-1α, IL-1β, IL-2, IL-3, IL-4, IL-5, IL-6, IL-7, IL-9 (P40), IL-10, IL-11, IL-12 and IL-13); CSF-type cytokines such as GM-CSF, G-CSF, M-CSF, LIF, EPO, TNF-α and TNF-β) ; interferons (such as IFN-α, IFN-β, IFN-γ); cytokines of the TGF-β family (such as TGF-β1, TGF-β2, TGF-β3, inhibin A, inhibin B, activin A, activin B); chemotactic factors (such as NAP-1, MCP-1, MIP-1α, MIP-1β, MIP-2, SISβ, SISδ, SISε, PF-4, PBP, γIP-10, MGSA); growth factors (such as EGF, TGF-α, aFGF, bFGF, KGF, PDGF-A, PDGF-B, PD-ECGF, INS, IGF-I, IGF-II, NGF-β); α-type intercrine cytokines (such as IL-8, GRO/MGSA, PF-4, PBP/CTAP/βTG, IP-10, MIP-2, KC, 9E3); and β-type intercrine cytokines (such as MCAF, ACT-2/PAT 744/G26, LD-78/PAT 464, RANTES, G26, I309, JE, TAC3, MIP-1α, B, CRG-2). A number of other cytokines are also known to those of skill in the art. The sources, characteristics, targets and effector activities of these cytokines have been described and, for many of the cytokines, the DNA sequences encoding the molecules are also known; see, e.g., Callard & Gearing, The Cytokine Facts Book (Academic Press, 1994) and the particular publications reviewed and/or cited therein, which are hereby incorporated by reference in their entirety. As referenced in catalogs such as The Cytokine Facts Book, many of the DNA and/or protein sequences encoding such cytokines are also generally available from sequence databases such as GENBANK (DNA); and SWISSPROT (protein). Typically, cloned DNA encoding such cytokines will already be available as plasmids, although it is also possible to synthesize polynucleotides encoding the cytokines based upon the published sequence information. Polynucleotides encoding the cytokines can also be obtained using polymerase chain reaction (PCR) methodology, as described in the art. [See, e.g., Mullis, 1987] The detection, purification, and characterization of cytokines, including assays for identifying new cytokines effective upon a given cell type, have also been described in a number of publications as well as the references referred to herein. [See, e.g., Lymphokines and Interferons, (Clemens, J. J. et al. eds., IRL Press 1987); and DeMaeyer, 1988].

“Transduction,” or “transducing” as used herein, are terms referring to the introduction of an exogenous polynucleotide into a host cell, irrespective of the method used for the insertion, which methods include well-known techniques such as transfection, lipofection, viral infection, transformation, and electroporation, as well as non-viral gene delivery techniques. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

A “replicon” refers to a polynucleotide comprising an origin of replication, generally referred to as an ori sequence, which allows for replication of the polynucleotide in an appropriate host cell. Examples include rep licons of a target cell into which a desired nucleic acid might integrate, e.g., nuclear and mitochondrial chromosomes, and extrachromosomal replicons such as plasmids.

An “individual” as used herein refers to a mammal, preferably a human.

“Treatment” or “therapy” as used herein refers to administering cells, other agents, or combinations thereof, to an individual, that are capable of eliciting a prophylactic, curative or other beneficial effect in the individual.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and posttranslational modification.

“Vasculature” or “vascular” are terms referring to the system of vessels carrying blood (as well as lymph fluids) throughout the mammalian body.

“Blood vessel” refers to any of the vessels of the mammalian vascular system, including arteries, arterioles, capillaries, venules, veins, sinuses, and vasa vasorum.

“Artery” refers to a vessel through which the blood passes away from the heart to the various parts of the body. The wall of an artery consists typically of an outer layer (adventitia) separated by an external elastic lamina from a middle layer (media) which is separated by an internal elastic lamina from an inner layer (intima). The adventitia is a layer of loose connective tissue which generally includes a network of micro vessels (vasa vasorum), Fibroblasts, and immune cells such as lymphocytes and macrophages. The media comprises circular layers of smooth muscle cells and elastic fibers. The intima is made up of a monolayer of endothelial cells overlying, in some instances, smooth muscle cells.

“Microvessel,” “microvascular” or “microvasculature,” as used herein, are terms referring to the arterioles, capillaries, venules, and adventitial microvessels. Microvessels generally comprise endothelial cells surrounded by one or a few layers of smooth muscle cells. Arteriole refers to a minute arterial branch, especially one just proximal to a capillary. Capillary refers to any one of the minute vessels that connect the arterioles and venules, forming a network in virtually all organs and tissues. Venules refer to any of the small vessels that collect blood from the capillary plexuses and join to form veins. “Adventitial microvessel” refers to microvessels that supply blood to the adventitia of larger blood vessels such as arteries. The network of these adventitial microvessels is commonly referred to as the vasa vasorum. Adventitial microvessels are believed to be supplied with blood from the lumen of the parent vessel (e.g., the artery) via small microvessels traversing the vessel intima and media. “Microvascular cell” refers to cells that make up the structure of microvessels.

“Endothelium” refers to the layer of cells (i.e., “endothelial cells”) that generally lines the cavities of the heart and blood vessels, as well as vessels of the lymphatic system. As described herein, vessels such as arteries can contain both endothelial cells and smooth muscle cells which are distinguishable in terms of origin, functionality, and attributes such as cell surface markers. For example, generally, endothelial cells derive from embryonic ectoderm, form the layer of cells that make up the endothelium, provide a non-thrombogenic surface, and can be readily distinguished using a number of well-known cell surface markers, including, by way of illustration, vWF, as exemplified below. Generally, smooth muscle cells derive from embryonic mesoderm, provide structure and contractile function for blood vessel walls, do not provide a non-thrombogenic surface, and can be readily distinguished using a number of well-known surface markers, including, by way of illustration, alpha-actin, as exemplified below.

“Proliferating” or “proliferation” are terms referring to growth by cell multiplication. Angiogenesis is the formation of new blood vessels which occurs during fetal development of the vascular system, as well in a wide range of normal and postnatal pathological processes such as wound repair, neoplasia, and inflammation.

References

The practice of the present invention will employ, unless otherwise indicated, conventional techniques of molecular biology, microbiology, recombinant DNA, and immunology, which are within the skill of the art. Such techniques are explained fully in the literature. See e.g., Molecular Cloning: A Laboratory Manual, (J. Sambrook et al., Cold Spring Harbor Laboratory, Cold Spring Harbor, N.Y., 1989); Current Protocols in Molecular Biology (F. Ausubel et al. eds., 1987 and updated); Essential Molecular Biology (T. Browned., IRL Press 1991); Gene Expression Technology (Goeddel ed., Academic Press 1991); Methods for Cloning and Analysis of Eucaryotic Genes (A. Bothwell et al. eds., Bartlett Publ. 1990); Gene Transfer and Expression (M. Kriegler, Stockton Press 1990); Recombinant DNA Methodology (R. Wu et al. eds., Academic Press 1989); PCR: A Practical Approach (M. McPherson et al., IRL Press at Oxford University Press 1991); Cell Culture for Biochemists (R. Adams ed., Elsevier Science Publishers 1990); Gene Transfer Vectors for Mammalian Cells (J. Miller & M. Calos eds., 1987); Mammalian Cell Biotechnology (M. Butler ed., 1991); Animal Cell Culture (J. Pollard et al. eds., Humana Press 1990); Culture of Animal Cells. 2nd Ed. (R. Freshney et al. eds., Alan R. Liss 1987); Flow Cytometry and Sorting (M. Melamed et al. eds., Wiley-Liss 1990); the series Methods in Enzymology (Academic Press, Inc.); Techniques in Immunocytochemistry, (G. Bullock & P. Petrusz eds., Academic Press 1982, 1983, 1985, 1989); Handbook of Experimental Immunology, (D. Weir & C. Blackwell, eds.); Cellular and Molecular Immunology (A. Abbas et al., W. B. Saunders Co. 1991, 1994); Current Protocols in Immunology (J. Coligan et al. eds. 1991); the series Annual Review of Immunology; the series Advances in Immunology; Oligonucleotide Synthesis (M. Gait ed., 1984); and Animal Cell Culture (R. Freshney ed., IRL Press 1987).

Additional references describing delivery and logistics of surgery which may be used in the methods of the present invention include the following: The Textbook of Interventional Cardiology. 2nd Ed. (E. Topol ed., W. B. Saunders Co. 1994); Rutherford, R. B., Vascular Surgery. 3rd Ed. (W. B. Saunders Co. 1989); Interventional Radiology, 2nd Edition (W. Castaneda-Zuniga & S. Tadavarthy eds., Williams & Wilkins 1992); Textbook of Respiratory Medicine, 2nd Ed. (J. Murray & J. Nadel eds., W. B. Saunders Co. 1994); and Textbook of Surgery, 14th Ed. (D. Sabiston, Jr., W. B. Saunders Co. 1991).

Additional references describing cell types found in the blood vessels, and the structure of the vasculature which may be used in the methods of the present invention include the following: W. Bloom & D. Fawcett, A Textbook of Histology, 10th Ed., (W. B. Saunders Co. 1975).

Additional references describing AAV vectors which may be used in the methods of the present invention include the following: Carter, B., Handbook of Parvoviruses, vol. I, pp. 169-228 (1990); Berns, Virology, pp. 1743-1764 (Raven Press 1990); Muzyczka, N., Current Topics in Microbiology and Immunology, 158: 92-129 (1992); and Kotin, R., Human Gene Therapy, 5: 793-801 (1994).

Bibliography of Articles Cited

Allen, J., PCT/US95/15892, filed Dec. 6, 1995.

Andree, C., et al., Proc. Natl. Acad. Sci. USA, 91 (25): 12188-12192 (1994).

Baillie, C., et al., Brit. J. Cancer, 72: 257-267 (1995).

Berns, Virology, pp. 1743-1764 (Raven Press 1990).

Betsholtz, C., et al., Nature, 320 (6064): 695-699 (1986).

Boshart, et al., Cell, 41: 521-530 (1985).

Brigstock, D., et al., Growth Factors, 4 (1): 45-52 (1990).

Callard, R. & Gearing, A., The Cytokine Facts Book, (Academic Press 1994).

Carter, B., Handbook of Parvoviruses, vol. I, pp. 169-228 (1990).

Carter, B., Current Opinions in Biotechnology, 3: 533-539 (1992).

Chang, M., et al., Science, 267: 518-522 (1995).

Clowes, M. M., et al., J. Clin Invest., 93: 644-651 (1994).

Conn, G., et al., Proc. Natl. Acad. Sci. USA, 87 (7): 2628-2632 (1990).

DeMaeyer, E., et al., Interferons and Other Regulatory Cytokines, (John Wiley & Sons 1988)

Derynck, R., et al., Nature, 316: 701-705 (1985).

Diaz-Flores, L., et al., Histol. Histopath, 9: 807-843 (1994).

Egan, M., et al., Nature, 358: 581-584 (1992).

Fields-Berry, et al., Proc. Natl. Acad. Sci. USA, 89: 693-697 (1992).

Flotte, T., et al., Am. J. Respir. Cell Mol. Biol. 7: 349-356 (1992).

Flotte (1), T., et al., J. Biol. Chem. 268 (5): 3781-3790 (1993).

Flotte (2), T., et al., Proc. Natl. Acad. Sci. USA, 93: 10163-10167 (1993).

Flotte, T., et al., Pediatric Pulmonology, Supplement 10, pp. 153-154 (1994).

Flotte (1), T., et al., WO 95/13365, published on May 18, 1995.

Flotte (2), T., et al., Gene Therapy, 2: 29-37 (1995).

Geary, R. L., et al., Circulation, 88 (4): I-81 (abstract) (1993).

Geary, R. L., et al, Human Gene Therapy, 5: 1211-1216 (1994).

Geary, R. L., et al., Atheroscler. Thromb. Vasc. Biol., 16: 34-43 (1996).

Geller, D., et al., Proc. Natl. Acad. Sci. USA, 90: 3491-3495 (1993).

Guzman, R., et al., Proc. Natl. Acad. Sci. USA, 91: 10732-10736 (1994).

Halbert, C., et al., J. Virol., 69 (3): 1473-1479 (1995).

Houck, K., et al., Mol. Endocrinol., 5 (12): 1806-1814 (1991).

Itoh, N., et al., Biochem. Biophys. Res. Comm., 169 (2): 680-685 (1990).

Jaffe et al., J. Clin. Invest., 52: 2745-2756 (1973).

Janssens, S., J. Biol. Chem., 267 (21): 14519-14522 (1992).

Johnsson, A., et al., EMBO J., 3 (5): 921-928 (1984).

Kaplitt, M., et al., Nature Genetics, 8: 148-154 (1994).

Kondo, S., et al., Biochimica et Biophysica Acta, 1243 (2): 195-202 (1995).

Kotin, R., Human Gene Therapy, 5: 793-801 (1994).

Lamb, P., et al., Mol. Cell. Biol., 6 (5): 1379-1385 (1986).

Lee, W., et al., Science, 235 (4794): 1394-1399 (1987).

Lemarchand, P., et al., Circulation Research, 72 (5): 1132-1138 (1993).

Lim, C. S., et al., J. Circ., 83: 2133-2136 (1991).

Lupton, S., WO 92/08796, filed Nov. 12, 1991.

Lupton, S., WO 94/28143, filed May 19, 1994.

Lymphokines and Interferons, (Clemens, J. J. et al. eds., IRL Press 1987).

Lynch, C. M.. Proc. Natl. Acad. Sci. USA, 89: 1138-1142 (1992).

Mullis & Faloona, Met. Enzymology, 155: 335 (1987).

Muzyczka, N., Current Topics in Microbiology and Immunologv, 158: 97-129 (1992).

Nabel, E. G., et al., Science, 244: 1342-1344 (1989).

Nabel, E. G., et al., Science, 249: 1285-1288 (1990).

Newman, K., et al., J. Clin. Invest., 96 (6): 2955-2965 (1995).

Ojeifo, J., et al., Cancer Research, 55: 2240-2244 (1995).

Plautz, G., et al., Circulation, 83: 578-583 (1991).

Robbins, P. D., et al, Nature, 346 (6285): 668-671 (1990).

Rome (1), J., et al., Arterioscler. Thromb., 14: 148-161 (1994).

Rome (2), J., et al., Human Gene Therapy, 5: 1249-1258 (1994).

Russell, D., et al., Proc. Natl. Acad. Sci. USA, 91: 8915-8919 (1994).

Schwartz, S. M., Physiol. Rev., 70 (4): 1177-1209 (1990).

Trempe, J., & Yang, Q., WO95/13392, published on May 18, 1995.

Von Der Leyen, H., et al., Proc. Natl Acad. Sci. USA, 92: 1137-1141 (1995).

Walsh, C., et al., Proc. Natl. Acad. Sci. USA, 89: 7257-7261 (1992).

Zwiebel, J. A., et al., Science, 243: 220-222 (1989).

All patents, patent applications, and publications mentioned herein, both supra and infra, are hereby incorporated herein by reference.

### DETAILED DESCRIPTION OF THE PREFERRED EMBODIMENTS

The invention described involves methods for transducing cells in blood vessels using recombinant AAV vectors. The transduction of cells in blood vessels in vivo presents problems not encountered in the transduction of other cells in vivo or in vitro.

AAV vectors are among a small number of recombinant virus vector systems which have been shown to have utility as in vivo gene transfer agents and thus are potentially of great importance for human gene therapy. AAV vectors are capable of high-frequency gene transfer and expression in a variety of cell lines ex vivo. [Carter, 1992; Egan, 1992; Flotte, 1992; Flotte (1), 1993; Flotte (2), 1993; Kaplitt, 1994; Kotin, 1994; Muzyczka, 1992; and Walsh, 1992] However, there have been very few studies of in vivo transduction using rAAV vectors in animal models. [See, e.g., Flotte (2), 1993; Kaplitt, 1994; and Kotin. 1994] AAV is a DNA parvirus often found in association with adenovirus infections of humans. AAV has not been shown to cause disease in man and is not a transforming or oncogenic virus. By contrast, most of the other proposed viral systems such as retroviruses, adenoviruses, herpesviruses, or poxviruses are disease-causing viruses. Indeed, greater than 85% of adults are believed to be seropositive for one of four AAV serotypes. AAV is also replication-defective, and can replicate only within the nucleus of cells simultaneously infected with a helper virus such as adenovirus, herpes virus, or in some cases poxviruses such as vaccinia. AAV is believed to be capable of infecting and replicating (albeit at different efficiencies) in virtually any cell line of human, simian or rodent origin if an appropriate helper is present. In addition, AAV has a clear advantage over retroviruses, especially in tissues such as the human airway epithelium where most cells are terminally differentiated and non-dividing, because AAV does not require active cell division for gene transfer. Thus AAV vectors are capable of transducing quiescent cells in situ and have recently been shown to be effective at transducing monkey pulmonary epithelium in vivo with the CFTR gene for cystic fibrosis. [See, e.g., Flotte, 1994] These studies have recently been carried into human trials approved by the NIH Recombinant DNA Advisory Committee. While AAV can transduce quiescent cells, recent work in primary fibroblast cultures suggests that AAV vectors preferentially transduce cells in S phase. [See, e.g., Russell, 1994] General reviews of AAV may be found in, e.g., Carter, 1990; Bems, 1990; Muzyczka, 1992; and Kotin, 1994.

AAV can be modified to create a vector for the delivery of heterologous genes. Preferably, such AAV vectors will have no wild-type coding sequences and will be incapable of replication, even in the presence of helper virus. Generally, the process of modification involves deleting all wild-type AAV coding sequences (rep and cap) so that only the inverted terminal repeat sequences (which are required in cis for vector replication) remain. A gene of interest can be inserted between the viral inverted terminal repeat sequences and then packaged.

The general principles of AAV vector construction have been recently reviewed. [See, e.g., Carter, 1992; Kotin, 1994; and Muzyczka, 1992] AAV vectors can be constructed in AAV recombinant plasmids by substituting portions of the AAV coding sequence with heterologous DNA to generate a vector plasmid. In the vector plasmid, the terminal (ITR) portions of the AAV sequence play an important role in cis for several functions including excision from the plasmid after transfection, replication of the vector genome and integration and rescue from a host cell genome. The vector can then be packaged into an AAV particle to generate an AAV transducing virus by transfection of the vector plasmid into cells that are: (1) infected by an appropriate helper virus such as adenovirus or herpesvirus, and (2) capable of providing AAV replication and encapsidation functions in trans (since these functions were deleted in construction of the vector plasmid). Several recent publications have described methods for generating high titers of recombinant AAV vectors. [See, e.g., Flotte (1) 1995; Trempe, 1995; and Allen, 1995]

In the methods of the present invention, recombinant AAV vector preparations can comprise AAV ITR regions and a transcription promoter operably linked to any gene of interest that is to be transduced to the recipient cell, including for example, detectable genes, selectable genes, and/or therapeutic genes. By way of illustration, in certain Examples below, the inventors used human placental alkaline phosphatase (AP) as a detectable gene. However, it will be clear to those of skill in the art, based on the teachings herein, that other recombinant AAV vectors containing one or more detectable, selectable, and/or therapeutic genes can be readily employed. Thus, for example, the methods of the present invention can employ rAAV vectors comprising (in place of or in addition to a detectable and/or selectable gene) a therapeutic gene that is used to alter the activity of the transduced recipient cell so that the recipient cell and/or its progeny have a beneficial effect on an individual receiving such cells. By way of illustration, a typical example would be the transduction of cells in a blood vessel with a therapeutic gene that enhances the level of a beneficial protein or other agent in the cell and/or its progeny, or that reduces the level of a deleterious protein or other agent in the cell and/or its progeny, or that provides resistance to a cytotoxic or other harmful agent

As another basic illustrative example, the present invention can be used to transduce cells in a blood vessel with a gene or genes that encode secreted proteins or that encode proteins involved in the secretion of other agents from the cell and/or its progeny, which secreted proteins or other agents have a beneficial effect on the recipient individual

As yet another illustrative example, the present invention can be used to transduce cells in a blood vessel with a polynucleotide, gene or genes that affect the interaction between a cell and/or its progeny and other cells in the recipient individual. By way of illustration, the therapeutic gene might render the transduced cells and/or their progeny more or less susceptible to activation by other cells, more or less resistant to a chemotherapeutic agent, or more or less resistant to an infectious agent such as a virus or a toxic agent such as a chemotherapeutic drug, to name just a few examples.

As those of skill in the art will appreciate, the present invention thus can be used to “deliver” any of a wide variety of genes to cells within the vasculature of a mammal, preferably a human. A few examples of specific therapeutic strategies taking advantage of the invention and its ability to transduce cells in blood vessels, especially microvessels, for recombinant gene delivery are outlined below. These additional examples are provided for purposes of further illustrating exemplary applications of the present invention. Numerous other genes can be delivered using the methods of the present invention, as will be clear to those of skill in the art. Thus, for example, using the methods of the invention, a number of genes can be expressed in the vasculature, especially microvessels, to increase cell growth of microvascular cells (e.g., promote angiogenesis) and/or to increase cell growth in a target tissue or organ supplied by the transduced microvessels. Such genes include, for example, human growth factors, platelet-derived growth factor (PDGF), vascular endothelial growth factors (VEGF), basic fibroblast growth factor (bFGF), transforming growth factor-beta (TGF-β), and epidermal growth factor (EGF). [See, e.g., Andree, 1994; Betsholtz, 1986; Brigstock, 1990; Conn, 1990; Derynck, 1985; Houck, 1991; Itoh, 1990; Johnsson, 1984; and Kondo, 1995]. The gene sequences for many such human growth factors are known and could be cloned into and expressed by rAAV vectors using standard cloning techniques as described in the references herein. Further, a large variety of other cytokines are referred to above, and known gene sequences for such cytokines can likewise be cloned and expressed by rAAV vectors using the methods of the invention. [See, e g, Callard. R. & Gearing, A., The Cytokine Facts Book, (Academic Press 1994)]

Conversely, a number of genes can be expressed in the vasculature, especially microvessels, to inhibit growth of microvascular cells and/or to decrease cell growth in a target tissue or organ supplied by transduced microvessels. For instance, growth factors have specific cell-surface receptors to which the respective growth factors bind to initiate their growth-promoting effects. For many growth factors and other cytokines mentioned above, the corresponding receptors have also been identified. [See, e.g., Callard, R. & Gearing, A., The Cytokine Facts Book, (Academic Press 1994)] The gene sequences for many such receptors are known and can be modified to create mutant receptors that, for example, bind growth factor without initiating a growth-promoting signal. This could also be achieved using soluble (e.g., secreted) forms of the mutant receptors. Thus, such mutant receptors could serve as antagonists for their respective growth factors; expression of such mutant receptors in cells in the vasculature, especially microvessels, which supply organs or tissues can inhibit growth of these cells and/or cells of the organ and tissue itself.

Numerous other inhibitors of cell growth that could be delivered to the vasculature, especially microvessels, using the methods of the invention include, for example, the nonphosphorylated form of the retinoblastoma (Rb) gene product, which inhibits cell cycle progression by binding to specific cellular transcription factors. [See, e.g., Chang, 1995; Lee, 1987; and Robbins, 1990] Also, for example, large artery cell proliferation could be inhibited by over-expressing genes such as endothelial cell nitric oxide synthase (which synthesizes nitric oxide, an inhibitor of vascular smooth muscle cell proliferation) in the vasa vasorum of a parent vessel. Thus, the methods of the invention could be used to reduce atherosclerosis and restenosis following arterial reconstructions. [See, e.g., Geller, 1993; Janssens, 1992; and Von Der Leyen, 1995] Further, numerous other genes can be expressed in the vasculature, especially microvessels, to promote cell death. As is further described below, expression of such “suicide” genes in transduced microvascular cells can result in the death of the transduced cells and death of adjacent cells in the target organ or tissue via a “bystander” effect. Such genes, for example, include herpes simplex virus thymidine kinase which encodes a protein rendering cells susceptibile to antiviral pro-drug ganciclovir. [See e.g., Guzman, 1994] In addition, programmed cell death (apoptosis) in cells in blood vessels, especially microvascular cells, can be promoted by over-expressing, for example, p53 tumor suppressor gene or other genes in the apoptosis pathways. [See, e.g., Lamb, 1986]

The rAAV vectors may also contain one or more detectable markers. A variety of such markers are known, including, by way of illustration, the bacterial beta-galactosidase (lacZ) gene; the human placental alkaline phosphatase (“AP”) gene and genes encoding various cellular surface markers which have been used as reporter molecules both in vitro and in vivo.

The rAAV vectors may also contain one or more selectable markers. As mentioned above, for applications involving gene therapy, it may also be advantageous for the rAAV vector to comprise a “suicide” gene that allows recipient cells in a blood vessel to be selectively eliminated at will. A suicide gene is a type of negative selectable marker gene that causes host cells to be inhibited or eliminated in the presence of the corresponding selective agent. Such suicide genes can thereby be used to selectively eliminate the host cells should that become necessary or desirable. [See, e.g., Lupton, 1991; and Lupton, 1994]

Recombinant AAV vectors can also comprise polynucleotides that do not encode proteins, including, e.g., polynucleotides encoding for antisense mRNA (the complement of mRNA) which can be used to block the translation of normal mRNA by forming a duplex with it, and polynucleotides that encode ribozymes (RNA catalysts).

The introduction of rAAV vectors by the methods of the present invention may involve use of any number of delivery techniques (both surgical and nonsurgical) which are available and well known in the art. Such delivery techniques, for example, include vascular catheterization, cannulization, injection, inhalation, inunction, topical, oral, percutaneous, intra-arterial, intravenous, and/or intraperitoneal administrations. Vectors can also be introduced by way of bioprostheses, including, by way of illustration, vascular grafts (PTFE and dacron), heart valves, intravascular stents, intravascular paving as well as other non-vascular prostheses. General techniques regarding delivery, frequency, composition and dosage ranges of vector solutions can be found in references such as those cited herein

As described herein, the methods of the present invention can be quite beneficial in a number of disease conditions in which blood vessels and/or angiogenesis play a role. For example, the ability to transduce cells in blood vessels, especially microvessels, of a specific tissue or organ would allow one to locally modify gene expression and to locally enhance the tissue or organ in a positive way. In addition, one can modify gene expression in cells in blood vessels to inhibit or enhance angiogenesis. There are many clinical situations associated with angiogenesis where the ability to limit or eliminate angiogenic potential using the methods of the present invention can be very beneficial. By way of illustration, both benign and malignant neoplasms typically require extensive blood supply to support growth and metastasis, so that inhibition of angiogenesis can be used to inhibit or eliminate these neoplasms. In fact, the malignant potential of many tumors can be directly correlated to the extent of microvascular content. [See, e.g., Baillie, 1995] Thus, while common approaches for treating neoplasms (including surgical excision, radiation therapy and chemotherapy) are either invasive or have potential for significant side effects, the methods of the invention can be used to target the blood supply of the tumor itself and, in a minimally invasive fashion, promote its elimination without systemic or permanent side effects.

Analogously, there are a number of other diseases where excessive blood supply is critical, which could therefore be benefited by inhibiting microvascular cells involved in angiogenesis using the gene delivery methods of the present invention. Examples include, but are not limited to, psoriasis; rheumatoid arthritis; ocular diseases such as diabetic retinopathy and retinitis; and congenital or acquired vascular malformations composed of arteries, veins or lymphatics, separately or in conjunction with each other.

Further, there are a number of clinical situations where promoting angiogenesis can be advantageous. By way of illustration, cardiac ischemia is characterized by insufficient blood and oxygen flow to the heart and is an example of a system that would be benefited by application of the present invention. By way of illustration, using the methods of the present invention, one can cannulate a coronary artery supplying the region of ischemia and deliver rAAV vectors (e g, comprising polynucleotides that can transduce and modify growth of microvascular cells in that area) to the regional microvasculature around the area of ischemia in order to induce new blood vessel growth into the area of ischemia.

Analogously, there are a number of other diseases in which lack of blood supply is critical, which can therefore be benefited by stimulating cells involved in angiogenesis using the gene delivery methods of the present invention. Examples include, but are not limited to, cerebral ischemia such as that which occurs during embolic stroke, certain ischemic nephropathies such as kidney parenchymal diseases, and cutaneous ulcers such as extremity ulcers.

In addition to modulating angiogenesis, the ability to transduce cells in blood vessels, especially microvessels, of an organ or tissue allows for gene expression that can be used to directly influence the tissue or organ supplied without necessarily affecting the microvessel itself. By way of illustration, when reconstructing vascular tissue such as coronary arteries blocked with atherosclerosis, scarring frequently results in a recurrent blockage (e.g., restenosis) at the site of reconstruction. Following coronary angioplasty or atherectomy, 30-60% of treated arteries develop restenosis. By using the methods of the invention to transduce cells in the vasa vasorum of an affected artery with, for example, an inhibitor of vascular smooth muscle cell proliferation, the atherosclerosis, scarring and subsequent restenosis following such arterial reconstructions can be reduced.

The examples presented below are provided as a further guide to the practitioner of ordinary skill in the art, and are not to be construed as limiting the invention in any way.

### EXAMPLES

### Example 1

### Generation of rAAV Preparations

For purposes of illustrating the present invention, the inventors prepared several rAAV vectors. For a number of the primary explant culture and in vivo studies described below, the inventors employed an adeno-associated virus-based vector called “ACAPSN” which comprises AAV ITR sequences, the human cytomegalovirus (CMV) promoter operably linked to the human placental alkaline phosphatase (AP) cDNA, the simian virus 40 promoter operably linked to the E. coli transposon Tn5 neomycin phosphotransferase (neo) gene, and a synthetic polyadenylation site.

Construction of ACAPSN vector

The construction of ACAPSN vector was as follows: the ITR sequences and plasmid backbone were derived from AAV-CFTR (which contains AAV2 nucleotides 1-145 comprising the left-hand ITR, the cystic fibrosis transmembrane regulator (CFTR) cDNA nucleotides 133 to 4573, a synthetic polyadenylation signal based on murine β-globin [See, e.g., Flotte (1), 1993] and AAV2 nucleotides 4490 to 4680 containing the right-hand ITR inserted in a plasmid backbone of pBR322 nucleotides 2295 (Nde1) to 4284 (Aat2)). Briefly, the AAV-CFTR vector was digested with Xho1 and SnaB1 and the ITRs and plasmid backbone were gel isolated. An Xho 1 to SnaB1 fragment containing a portion of the CMV promoter (nucleotides −671 to −464) [See, e.g., Boshart, 1985] was gel isolated and ligated to the ITR plasmid backbone fragment derived from AAV-CFTR to generate “pAAV-CMV (SnaB1).” Next, an Spe1to SnaB1 fragment containing the synthetic polyadenylation signal was inserted into Spe1/SnaB1 digested pAAV-CMV (SnaB1) to generate “pAAV-CMV (Spe1)-spA.” The pAAV-CMV (Spe1)-spA vector contains nucleotides −671 to −584 of the CMV promoter. Next, the human placental alkaline phosphatase cDNA sequence linked to the Simian virus 40 promoter driving the E.coli neomycin gene was isolated from LAPSN [See, e.g., Lynch, 1992] as an Spe1to Nhe1 fragment and inserted into pAAV-CMV (Spe 1)-spA (which had been linearized with Spe1) to create “pAAV-APSN.” An Spe1to Nhe1 fragment containing CMV promoter nucleotides −585 to +71 was inserted into Spe1linearized pAAV-APSN to generate vector “ACAPSN.”

Construction of AAV-CMV-AP vector

The inventors also employed a second adeno-associated virus-based vector (“AAV-CMV-AP”) in their primary explant culture studies. The construction of vector AAV-CMV-AP was as follows: the AAV vector pTRF46 [See, e.g., Flotte (1), 1993] was restriction endonuclease digested with Hind3 and Asp718 and the fragment containing the ITR sequences, synthetic polyadenylation site (and including the pBR322 derived plasmid backbone) was gel isolated. A neomycin phosphotransferase gene (neo) which had been engineered with a Kozak consensus eukaryotic translation initiation sequence was PCR amplified using primers that gave a Hind3site at the 5′ end and an Asp718 site at the 3′ end of the neo sequences. The Hind3/Asp718 digested neo PCR product was ligated to the pTRF46 derived fragment to generate “pAAVneoBR.” The AAV and neo sequences were excised from the pBR322 derived plasmid backbone at the Bg12 sites flanking the ITRs and subcloned into a Bluescript vector in which the multiple cloning site was substituted with a Bg12 linker to create “pAAVneo.” A Sal1 fragment containing the human placental alkaline phosphatase cDNA sequence was isolated from the retroviral vector DAP. [See, e.g., Fields-Berry, 1992] A 2 base pair fill-in was performed and the fragment inserted into pAAVneo which had been linearized with BarnH1 and partially filled-in to accept the insert. The resulting intermediate construct “pAAVneoAP” was digested with Hind3to remove the neo sequences. The construct was religated to create “pAAVAP.” A 2 base pair fill-in was performed on an Spe1to Nhe1 fragment containing the CMV immediate early promoter sequences [See, e.g., Boshart, 1985] (from nucleotide −583 to +71) and then was inserted into pAAVAP which had been linearized with Hind3and also partially filled-in. The resulting construct was vector “AAV-CMV-AP.”

Packaging of rAAV particles

Large scale packaging of rAAV particles was performed as previously described. [See, e.g., Flotte (1). 1995; and Flotte (2). 1995] According to these packaging protocols, equal amounts of packaging plasmid pRS5 and rAAV vector (i.e., either ACAPSN or AAV-CMV-AP) were cotransfected into 293 cells which had been infected with helper virus Ad5 (m.o.i.=5). After an incubation of 72 hours at 37° C., the cells were harvested and lysed. The virus particles were purified by cesium chloride gradient, fractionated and dialyzed against Ringer's balanced salt solution. The vector preparation was then heat treated to kill any residual adenovirus. Although the possibility of adenovirus contamination is believed to be remote, the preparation was handled using Biosafety Level 2 precautions appropriate for adenovirus (class-2 pathogen) as outlined in publication No.(CDC) 93-8395 of the U.S. Department of Health and Human Services entitled “Biosafety in Microbiological and Biomedical Laboratories” and “NIH Guidelines for Research Involving Recombinant DNA Molecules,” Federal Register 1994 , 59: 34496-34547.

### Example 2 (a)

### Transduction of Smooth Muscle Cells in Human Primary Explant Cultures Using the AAV-CMV-AP Vector

In order to investigate the delivery of rAAV vectors to a variety of cell types typically found in blood vessels, the inventors examined the transduction of smooth muscle cells in human primary explant cultures using the AAV-CMV-AP vector.

Human smooth muscle cells were derived from a biopsy sample by enzymatic digestion using procedures as described in the art. Primary explant cultures were characterized and confirmed to contain predominantly smooth muscle cells using immunocytochemical staining for alpha-actin, as described in Example 6 below. Cells were seeded at a density of 1×10⁵ cells/12 wells such that the cultures were semi-confluent (50% coverage of the dish) 24 hours later at the time of exposure to AAV-CMV-AP vector. Cells were exposed to 10 μl of AAV-CMV-AP vector in a total volume of 1 ml complete medium (containing a final concentration of 10% fetal bovine serum) for 24 hours. Smooth muscle cells were transduced with three independent preparations of AAV-CMV-AP vector. The vector preparations were estimated by slot-blot analysis of DNA genomes to contain on the order of 10⁹ particles per ml. Transductions were thus performed with approximately 100 particles per cell. Virus was removed from the cells at the end of the 24 hour exposure period and the cells were cultured in fresh medium for an additional 24 hours.

The cells were harvested using trypsin and 2.5×10⁴ cells were transferred onto glass slides using a cytospin. The slides were allowed to air dry, and the cells were then fixed in 0.5% glutaraldehyde in PBS. The cells were washed in three changes of PBS and heat treated for 30 minutes at 65° C. to inactivate any endogenous alkaline phosphatase activity. The cells were incubated in 5-bromo-4-chloro-3-indolyl phosphate (X-phos, 0.1 mg/ml, Boehringer Mannheim) and nitroblue tetrazolium (NBT, 1 mg/ml, Boehringer Mannheim, in Buffer 3 [100 mM Tris, 100 mM NaCl, 50 mM MgCl₂- pH 9.5]) for 3 hours at room temperature in the dark, which resulted in dark purple/black staining of cells expressing alkaline phosphatase. The slides were then rinsed in PBS and coverslipped using aqueous mounting medium. The percent of cells expressing alkaline phosphatase was determined by counting cells under light microscopy in at least two fields using a eye piece grid. The mean transduction frequency from the three independent infections was approximately 5.37%.

### Example 2 (b)

### Transduction of Smooth Muscle Cells in Rat Primary Explant Cultures Using the AAV-CMV-AP Vector

In order to further investigate the delivery of rAAV vectors to a variety of species, the inventors also examined the transduction of smooth muscle cells in rat primary explant cultures using the AAV-CMV-AP vector.

Rat smooth muscle cell cultures were derived from Fischer 344 rat aorta by enzymatic digestion and confirmed to contain predominantly smooth muscle cells using immunocytochemical staining for alpha-actin, as described above. [See, e.g. Lynch, 1992] Cells were transduced with AAV-CMV-AP vector at 100 particles per cell, exactly as described for human smooth muscle cell transductions in Example 2 (a). The mean frequency of the three transductions was approximately 0.032%.

The rodent (rat) smooth muscle cells were transduced at a 100-fold lower frequency than the primate (human and macaque) vascular cell types tested The decreased susceptibility of rodent cells to transduction with human adeno-associated virus derived vectors may indicate some species specificity of AAV.

### Example 2 (c)

### Transduction of Smooth Muscle Cells in Rabbit Primary Explant Cultures Using the AAV-CMV-AP Vector

As part of their investigations, the inventors also examined the transduction of smooth muscle cells in rabbit primary explant cultures using the AAV-CMV-AP vector. Rabbit smooth muscle cells were derived from rabbit aorta by enzymatic digestion, characterized and confirmed to contain predominantly smooth muscle cells using immunocytochemical staining for alpha-actin, as described above. Cells were transduced with a single preparation of AAV-CMV-AP vector at 100 particles per cell, as described in Example 2 (a). Human and rat smooth muscle cells were also transduced for comparison purposes. Rabbit smooth muscle cells were transduced at a frequency of approximately 10.26%, or twice the frequency of human smooth muscle cell (approximately 5.41%) in the same study. Rat smooth muscle cells were again transduced at a frequency two orders of magnitude lower (approximately 0.016%).

### Example 2 (d)

### Transduction of Smooth Muscle Cells in Macaque Primary Explant Cultures Using the ACAPSN Vector

Next, the inventors also examined the transduction of smooth muscle cells in monkey primary explant cultures using the ACAPSN vector. Smooth muscle cells were derived from a Macaca fascicularis biopsy sample by enzymatic digestion, characterized and confirmed to contain predominantly smooth muscle cells by immunocytochemical staining for alpha-actin, as described above. Cells were transduced with ACAPSN vector as described for human smooth muscle cell transductions in Example 2 (a), except that approximately 1000 particles per cell were used. The mean frequency of the transduction was approximately 13.19%

### Example 2 (e)

### Transduction of Umbilical Vein Endothelial Cells in Human Primary Explant Cultures Using the ACAPSN Vector

In order to further investigate delivery of rAAV vectors into cell types typically found in blood vessels, the inventors also examined the transduction of endothelial cells in human primary explant cultures using the ACAPSN vector. Human umbilical vein endothelial cells were derived from a biopsy sample following standard protocols for preparation of primary umbilical vein endothelial cell explant cultures. [See, e.g., Jaffe, 1973] Primary explant cultures were characterized and confirmed to contain predominantly endothelial cells by immunocytochemical staining for von Willebrand's Factor (vWF), as described in Example 6 below. Cells were transduced with the ACAPSN vector as described for human smooth muscle cell transductions in Example 2 (a), except that approximately 1000 particles per cell were used. The mean frequency of the transduction was approximately 4.56%.

### Example 2 (f)

### Transduction of Microvascular Endothelial Cells in Human Primary Explant

Cultures Using the ACAPSN Vector Additionally, the inventors also examined the transduction of microvascular endothelial cells in human primary explant cultures using the ACAPSN vector. Human microvascular endothelial cells were derived from omentum (fatty tissue) using enzymatic digestion and sequential plating on fibronectin coated plastic cultureware to remove contaminating fibroblasts as described in the art. Primary explant cultures were characterized and confirmed to contain predominantly endothelial cells by immunocytochemical staining for vWF, as described above. Cells were transduced with the ACAPSN vector as described for human smooth muscle cell transductions in Example 2 (a), except that approximately 1000 particles per cell were used. The mean frequency of the transduction was approximately 12.64%

### Example 2 (g)

### Transduction of Microvascular Endothelial Cells in Macaque Primary Explant Cultures Using the ACAPSN Vector

The inventors also examined the transduction of microvascular endothelial cells in monkey primary explant cultures using the ACAPSN vector. Macaque microvascular endothelial cells were derived from omentum and characterized and confirmed to contain predominantly endothelial cells using immunocytochemical staining for vWF as described above. Cells were transduced with the ACAPSN vector as described for human smooth muscle cell transductions in Example 2 (a), except that approximately 1000 particles per cell were used. The mean frequency of the transduction was approximately 4.93%.

Summary of Primary Explant Culture Studies

As described above, the inventors conducted a series of investigations to explore the delivery of rAAV vectors to cells typically found in blood vessels (e.g., smooth muscle cells, large vessel endothelial cells, microvascular endothelial cells) from a variety of different species. It is important to emphasize that while these preliminary studies were conducted “in vitro,” the inventors utilized primary explant cultures, which should be distinguished from immortalized cell cultures. Primary explant cultures, which have a finite lifespan, are believed to be far more representative of cells in vivo than immortalized cell culture lines which have generally been transformed with an oncogene so that they may grow indefinitely. Furthermore, it has been reported that while high levels of transduction have been reported with a number of cell culture lines, AAV vectors transduce primary cells much less efficiently than immortalized cells. [See, e.g., Halbert, 1995] Thus, the importance of using primary cells to evaluate AAV vectors for in vivo gene therapy applications is underscored.

These primary explant culture studies indicated that rAAV vectors can transfer and express a heterologous gene in a variety of the cell types typically found in blood vessels from a number of different species, including non-human primates and humans.

### Example 3

### In Vivo Transduction of Microvascular Cells in Primates Using ACAPSN Introduced by Intraluminal Delivery Without Pre-Treatment

The methods of the present invention are illustrated herein by in vivo studies conducted with non-human primates. In the following examples, the ACAPSN vector was infused into a segment of a peripheral artery in cynomolgus monkeys with established atherosclerosis. As further described below, the transductions using the methods of the present invention were evaluated under a variety of conditions by varying the state of the artery being treated and/or the delivery method employed. In this Example, ACAPSN was delivered by intraluminal infusion to unperturbed femoral arteries.

Two atherosclerotic cynomolgus monkeys (Macaca fascicularis), weighing approximately 5 kg were selected from the breeding colony at the Comparative Medicine Clinical Research Center of The Bowman Gray School of Medicine. The animals had consumed an atherogenic diet for varying periods of time and continued consuming this diet during the period of study. Aspirin was given three days prior to surgery (20 mg/kg, orally) for its anti-platelet effect. The animals received 5-bromo2′-deoxyuridine-5′-monophosphate (BrdU, 30 mg/kg, i.m., in saline [30 mg/ml], Boehringer Mannheim Inc., Indianapolis, Ind.) one and sixteen hours before surgery, to label proliferating cells for further studies (described in Example 8 below). The monkeys were sedated with ketamine hydrochloride (10-15 mg/kg i.m.), intubated and anesthetized with halothane gas (4% to effect). Blood was drawn for baseline serum, hematology and chemistries.

Left and right femoral arteries were exposed in the thigh using sterile technique and controlled near the groin and 4 cm distally. The animals were then anticoagulated with heparin (300 mg/kg i.v., Elkins-Sinn, Inc., Cherry Hill, N.J.) and a flexible cannula (20 gauge i.v. catheter) was inserted into each arterial side branch. These arteries were temporarily occluded sequentially and flushed with I ml sterile Ringer's solution. An ACAPSN vector preparation (250-500 μl), generated according to the procedures of Example 1. was infused into the left artery. The transduction was conducted using an ACAPSN preparation with a concentration of 3×10⁹ DNAse-resistant vector particles per ml (DNAse-resistance being characteristic of encapsidated vector particles). A control (250-500 μl of Lactate Ringer's solution, “LRS”) was infused into the right artery. After thirty minutes, ACAPSN vector preparation and LRS control were aspirated from the left and right arteries, respectively. The side branches were then ligated, and blood flow was re-established.

At the completion of the vector and control incubations, the wounds were closed and the animals were returned to single-animal cages for recovery. Animals remained quarantined for 48 hours and observed closely for any signs of viral illness such as conjunctivitis, cough and diarrhea. The animals were then anesthetized and the treated left and right arteries were surgically resected and fixed in 10% formalin for analysis. All animal care and procedures were performed in accordance with state and federal laws. Animal protocols were approved by the Bowman Gray School of Medicine Animal Care and Use Committee and conformed to guidelines set forth by the National Institutes of Health in publication No. 8623, Guide for the Care and Use of Laboratory Animals.

The formalin-fixed arterial segments were stained for human placental alkaline phosphatase to localize transduced cells expressing the ACAPSN vector as follows: tissues were washed in three changes of phosphate buffered saline (PBS, 10 ml, 1 hr/wash) and heated in PBS for thirty minutes in a 65° C. water bath to inactivate any endogenous alkaline phosphatase activity. Samples were then incubated in a solution of 5-bromo-4-chloro-3-indolyl phosphate (X-phos, 0.1 mg/ml, Boehringer Mannheim) and nitro-blue tetrazolium (NBT, 1 mg/ml, Boehringer Mannheim, in Buffer 3 [100 mM Tris, 100 mM NaCl, 50 mM MgCl₂-pH 9.5]) overnight at room temperature in the dark to detect alkaline phosphatase activity (dark purple/black staining). Samples were then washed in PBS and embedded in paraffin for tissue sectioning. Positive controls were included with each AP assay and were either: (1) cytospins of cultured smooth muscle cells transduced with the ACAPSN vector and fixed with 10% formalin; or (2) cultured smooth muscle cells transduced with the ACAPSN vector, pelleted, fixed with 10% formalin, embedded in paraffin and sectioned Negative controls were included for each animal and were either: (1) sections cut from arteries infused with Lactate Ringer's Solution; or (2) sections cut from arteries remote from the site of ACAPSN treatment.

Examination of the treated femoral artery tissue was conducted in a manner designed to systematically sample sections at regular intervals along the length of the treated vessel. Generally, each treated portion (approximately 1.5 inches in length) was sectioned into six rings. Every third ring was embedded in OCT and frozen. The first, second, fourth and fifth rings were fixed in 10% formalin, stained for AP activity (as described above), and then embedded in paraffin for sectioning (into sections approximately two cell layers thick, two to three sections per ring).

It should be emphasized however, that the samples examined collectively represent only a very small fraction (probably less than 1%) of the treated vessel tissue. Accordingly, positive results observed in the limited number of random sections examined would be expected to reflect expression throughout the tissue, while less frequent transduction events may have gone unobserved because of the limited number of samples examined.

In the case of the unperturbed femoral arteries, no expression events were observed in the sections examined. Due to the limited sampling, however, a low level of expression cannot be ruled out. It should also be noted that these femoral arteries had been scarred from previous bleeding (due to multiple blood draws in the femoral location) and thus fewer microvessels would be expected to be present because of peri-arterial scarring. Subsequent in vivo studies utilized carotid arteries.

### Example 4

### In Vivo Transduction of Microvascular Cells in Primates Using ACAPSN Introduced by Intraluminal Delivery Following Denudation Pre-Treatment

In order to determine whether endothelial cells function as a barrier to rAAV gene delivery deeper into the artery vessel wall, the ACAPSN vector was delivered by intraluminal infusion to carotid arteries which had been denuded of endothelium.

Two animals received intraluminal delivery subsequent to acute balloon denudation pre-treatment as follows. An ACAPSN vector preparation was generated according to the procedures of Example 1 The transduction was conducted using an ACAPSN preparation with a concentration of 3×10⁹ DNAse-resistant particles per ml. The animals were generally prepped, selected and maintained as described in Example 3, with the following exceptions: (1) the carotid arteries were studied in this Example; and (2) prior to intraluminal delivery of the ACAPSN vector and control (to the left and right carotids, respectively), a denudation pre-treatment was given as follows. The carotid arteries were denuded of endothelium using a 3F Fogarty balloon embolectomy catheter (V. Mueller Inc., McGaw Park, Ill.) which was passed (3 times) 3 cm into each carotid artery, inflated, retrieved under gentle tension and then removed.

The subsequent handling and preparation of animals and tissue followed the procedures outlined in Example 3, with the following three exceptions: the treated arteries were removed after 60 hours of quarantine; and (2) animals were then sedated with ketamine (15 mg/kg, i.m.), heparinized (300 units/kg, i.v.), and transported to the necropsy suite where blood was drawn for hematologic and chemistry assessments. After an overdose of sodium pentobarbital (100 mg/kg, i.v.) the animals were exsanguinated while infusing Ringer's solution (approximately 750 cc) at 100 mm Hg. The right and left carotid arteries were perfusion-fixed in 10% formalin and then removed for analysis. (3) Additionally, examination of the treated carotid artery tissue was conducted in a manner designed to systematically sample sections at regular intervals along the length of the treated vessel, both distal to and proximal to the site of injection. Generally, each treated portion (approximately 2 inches in length) was sectioned into nine rings and every third ring was embedded in paraffin. The first, second, fourth, fifth, seventh and eighth rings were frozen and two to three sections (approximately two cell layers thick) of each of these rings was then sampled and stained for AP activity (as described in Example 3).

In the case of the denuded carotid arteries, expression was observed in the microvascular endothelium in one of the two carotid arteries exposed to the rAAV vector, but was not observed in either control artery. Expression was readily localizable to the microvascular endothelium which forms a clearly definable structure in the adventitia.

### Example 5

### In Vivo Transduction of Microvascular Cells in Primates Using ACAPSN Introduced by Intraluminal Delivery Following Denudation and Focal Over-Distention Pre-Treatment

In order to further evaluate the effect of enhancing access to the outer adventitial layer of the vessel wall (where the microvessels primarily reside), the ACAPSN vector was delivered by intraluminal infusion to carotid arteries which had been gently denuded and focally distended.

Two animals received intraluminal delivery subsequent to gentle endothelial cell denudation and focal balloon over-distention pre-treatment as follows. An ACAPSN vector preparation was generated according to the procedures of Example 1. The transduction was conducted using an ACAPSN preparation with a concentration of 1×10¹⁰ DNAse-resistant particles per ml. The animals were generally prepped, selected and maintained as described in Example 3, with the following exceptions: (1) the carotid arteries were studied in this Example; and (2) prior to intraluminal delivery of the ACAPSN vector and control (to the left and right carotids, respectively), the carotid arteries were gently denuded of endothelium and subjected to focal over-distention. This pre-treatment was performed as follows: a 3F Fogarty balloon embolectomy catheter (V. Mueller Inc., McGaw Park, Ill.) was passed (once) 4 cm into the carotid artery, inflated and withdrawn towards the catheter insertion site using very minimal tension to remove the endothelium. The arterial segment directly adjacent to the catheter insertion site was then focally overdistended by over inflation of the balloon (in a 0.5 cm length of the carotid artery) and the balloon was then removed.

The subsequent handling and preparation of animals and tissue followed the procedures outlined in Example 4, with the exception that the treated arteries were removed after 48 hours of quarantine.

In the case of the gently denuded and focally over-distended carotid arteries, expression was observed in the endothelium of a small number of microvessels in the adventitia of both carotid arteries exposed to rAAV vector, but was not observed in either control artery There was also some lower level expression observed in adventitial cells (that were not clearly identified as endothelial cells) of both carotid arteries exposed to rAAV vector, but was not observed in either control artery.

### Example 6

### In Vivo Transduction of Proliferating Microvascular Cells in Primates Using ACAPSN Introduced by Adventitial Injection Delivery Following Prior Balloon Injury Pre-Treatment

In order to further investigate the effect of enhancing access to the adventitial layer of the vessel wall, the ACAPSN vector was also delivered by injection directly into the adventitia of a carotid artery that had been stimulated several days prior to delivery. Specifically, the artery was stimulated by repeated intraluminal passage of an inflated balloon catheter five days prior to delivery of ACAPSN, which would be expected to increase the rate of cellular proliferation of the vessel wall prior to delivery of the vector. From previous timecourse studies, the maximum proliferation of cells throughout the artery wall is induced within 4-7 days of balloon injury. [See, e.g., Geary, 1996] Thus, prior balloon injured vessels are expected to mimic physiological characteristics of proliferating vessels, such as occur during angiogenesis and following arterio-angioplasty or other forms of vascular reconstruction.

One animal received adventitial injection delivery subsequent to a prior balloon injury pre-treatment as follows. An ACAPSN vector preparation was generated according to the procedures of Example 1. The transduction was conducted using an ACAPSN preparation with a concentration of 3×10⁹ DNAse-resistant particles per ml. The animal was generally prepped, selected, and maintained as described in Example 3, with the following four exceptions: (1) the carotid arteries were studied in this Example; (2) both carotids received the ACAPSN vector; and (3) delivery occurred by direct adventitial injection. Adventitial injection was performed by the procedure used for intraluminal delivery as described in Example 3, with the following exception: instead of placing a cannula in the lumen, 250 μl of the ACAPSN vector preparation was injected per side, directly into the subadventitial plane, using a 1 cc syringe and 27 gauge needle. (4) Additionally, five days prior to delivery of the vector to the left and right carotids, the right carotid artery was subjected to balloon injury by three intralumina passages of an inflated 3F Fogarty balloon catheter (V. Mueller Inc., McGaw Park, Ill.) under moderate tension. The left carotid was previously unmanipulated.

The subsequent handling and preparation of animals and tissue followed the procedures outlined in Example 4, with the following exceptions: (1) the treated arteries were removed after 72 hours of quarantine; and (2) immuunocytochemical analysis was performed on tissue taken from the right carotid as follows.

To positively identify the particular cell type expressing ACAPSN in the adventitia, frozen formalin-fixed sections from the right carotid were cut and immunostained with antibodies specific to smooth muscle cell (alpha-actin, Boehringer Mannheim), endothelial cell (vWF, DAKO), macrophage (CD-68, DAKO) and lymphocyte (CD-3, DAKO). Primary antibodies were localized with appropriate biotinylated secondary antibodies (Vector Laboratories Inc.) and tertiary avidin-biotin-peroxidase staining (Vector Laboratories Inc.). Sections were counterstained with hematoxylin and examined by standard light-microscopy. For the right carotid, much of the AP activity co-localized with cells staining for either endothelial cell or smooth muscle cell markers. A minority of the AP activity co-localized with cells staining for a macrophage marker. A number of AP positive cells could not be positively identified with these cell type-specific antibodies.

In the case of delivery by adventitial injection to a carotid artery that had been previously stimulated, a very high frequency of expression was observed in cells identified histologically within adventitial microvessels. For this artery, expression was also observed in a significant number of other cells in the adventitia at the region of injection; as described above, these were not positively identified with cell-type specific antibodies and could possibly be, for example, fibroblasts, lymphocytes, other leukocytes, and nerve cells. In the unmanipulated control artery, a small number of AP positive cells was observed, and these few cells were predominantly associated with structures histologically consistent with microvessels. These results would lead to an expectation that in other situations where the proliferation of cells in a blood vessel is present or enhanced, one could also achieve high frequency transduction using the methods of the invention

### Example 7

### In Vivo Transduction of Proliferating Microvascular Cells in Primates Using ACAPSN Introduced by Intraluminal Delivery Following Prior Balloon Injury Pre-Treatment

As described above, adventitial microvessels are believed to be supplied with blood from the lumen of the parent vessel (e.g., the artery) via small microvessels traversing the vessel media. This study was designed to demonstrate that introduction of an rAAV vector into the adventitia can also be achieved indirectly, by injection directly into the lumen of an artery that had been pre-treated by prior balloon injury. As in Example 6, the artery was stimulated by repeated intraluminal passage of a large inflated balloon catheter five days prior to delivery of ACAPSN, which would be expected to increase the rate of cellular proliferation of the vessel wall prior to delivery of the vector.

One animal received intraluminal delivery subsequent to a prior balloon injury pre-treatment as follows. An ACAPSN vector preparation was generated according to the procedures of Example 1. The transduction was conducted using an ACAPSN preparation with a concentration of 1×10¹⁰ DNAse-resistant particles per ml. The animal was generally prepped, selected and maintained as described in Example 6, with the following exception: delivery occurred by intraluminal delivery as described in Example 3. Thus, in this Example, (1) both carotids received ACAPSN; and (2) five days prior to delivery of the vector to the left and right carotids, the right carotid artery was subjected to balloon injury by three intraluminal passages of an inflated 3F Fogarty balloon catheter (V. Mueller Inc., McGaw Park, Ill.) under moderate tension. The left carotid was previously unmanipulated

The subsequent handling and preparation of animals and tissue followed the procedures outlined in Example 4, with the exception that the treated arteries were removed after 72 hours of quarantine.

In the case of intraluminal delivery to a carotid artery that had been previously stimulated, expression was observed in cells identified histologically within adventitial microvessels (but was not observed in the control artery). Although the frequency of AP positive cells was not as high as that observed with injection directly into the adventitia, these results clearly demonstrated that introduction into the adventitia can be achieved by injection into the lumen of the blood vessel.

Summary of in vivo studies

These in vivo studies demonstrated, for the first time, that rAAV vectors could be successfully used to transduce cells in blood vessels in vivo.

Significant levels of transduction were observed in arteries that had been treated by prior balloon injury. As discussed above, such arteries would be expected to mimic physiological characteristics of proliferating vessels, such as occur during angiogenesis. These results would lead to an expectation that in other situations where vessel proliferation is enhanced, one could also achieve high frequency transduction using the methods of the invention.

Further, these in vivo studies demonstrated transduction of cells in atherosclerotic primate arteries, a diseased artery model that is most representative of the atherosclerosis disease state found in humans. Although the atherosclerotic state of the arteries in these macaques was not severe, it should be noted that physical access to various cells of a blood vessel is expected to be more difficult to achieve in even mildly atherosclerotic vessels because of the presence of, for example, plaques, fatty deposits, and/or thickened smooth muscle cell layers in the blood vessel. Thus, the ability to successfully transduce cells in blood vessels in an atherosclerotic macaque model using the methods of the present invention indicates that transduction of atherosclerotic vessels can be achieved and that even higher levels of transduction may be achieved in non-atherosclerotic vessels in which access conditions are less stringent.

### Example 8

### Evaluation of Post-Injury Cell Proliferation Using Antibody Staining

As described above, all animals were injected with BrdU one and sixteen hours prior to euthanasia, to allow for determination of cell proliferation in histological sections demonstrating positive ACAPSN staining. It is believed that transduction is most efficient for cells most actively proliferating.

Selected unstained deparaffinized sections (5 micron thick) from treated arteries will be exposed to a monoclonal antibody against BrdU (Boehringer Mannheim) and then localized with biotinylated secondary antibodies and avidin-biotin-peroxidase tertiary antibody staining. Proliferating cells will be defined as nuclei with brown antibody staining. The BrdU data may provide further confirmation that rAAV uptake and expression is associated with cells that are proliferating, such as those associated with angiogenesis. Further, angiogenesis models such as ocular microvascular cells or egg-yolk sac models can be used to quantify similar results in primary tissue in primary cells.

1. A method of transducing a cell in a blood vessel of an individual, comprising introducing a recombinant adeno-associated viral (rAAV) vector to a blood vessel of said individual in vivo.

2. A method of transducing a cell in a blood vessel according to claim 1, wherein said rAAV vector comprises a detectable marker gene.

3. A method of transducing a cell in a blood vessel according to claim 1, wherein said rAAV vector comprises a selectable marker gene.

4. A method of transducing a cell in a blood vessel according to claim 1, wherein said rAAV vector comprises a therapeutic gene.

5. A method of transducing a cell in a blood vessel according to claim 1, wherein said blood vessel is a micro vessel selected from the group consisting of arteriole, capillary, venule, and adventitial microvessel.

6. A method of transducing a cell in a blood vessel according to claim 5, wherein said blood vessel is an adventitial microvessel.

7. A method of transducing a cell in a blood vessel according to claim 1, wherein said blood vessel is a microvessel and said cell is undergoing proliferation

8. A method of transducing a cell in a blood vessel according to claim 1, wherein said cell is a primate cell.

9. A method of transducing a cell in a blood vessel according to claim 8, wherein said c ell is a human cell.

10. A method of transducing a cell in a blood vessel according to claim 1, wherein said cell is a proliferating cell.

11. A method of transducing a cell in a blood vessel according to claim 10, wherein said cell is a proliferating microvascular cell.

12. A method of transducing a cell in a blood vessel according to claim 1, wherein said cell is a microvascular cell.

13. A method of transducing a cell in a blood vessel according to claim 12, wherein said cell is a microvascular endothelial cell.

14. A method of transducing a cell in a blood vessel according to claim 1, wherein said rAAV vector is introduced into the adventitia of an artery of said individual.

15. A transduced microvascular cell produced by introducing a recombinant adeno-associated viral (rAAV) vector to said microvascular cell.

16. A method for treating an individual for a disease condition, comprising transducing a cell in a blood vessel of said individual according to the method of claim 4.
